(19) United States
(12) Patent Application Publication
(10) Pub. No.: US 2017/0087516 A1
(43) Pub. Date: Mar. 30, 2017

(54) ACTIVITY REPLENISHMENT AND IN SITU ACTIVATION FOR ENZYMATIC CO2 CAPTURE PACKED REACTOR

(71) Applicant: C02 Solutions Inc., Quebec (CA)

(72) Inventors: Eric Madore, Quebec (CA); Sylvie Fradette, Pintendre (CA); Jeremy Lawson, Montreal (CA)

(21) Appl. No.: 15/373,671

(22) Filed: Dec. 9, 2016

Related U.S. Application Data
(63) Continuation of application No. 14/401,609, filed on Nov. 17, 2014, now abandoned.

(30) Foreign Application Priority Data
May 17, 2012 (CA) 2778095

(51) Int. Cl. B01D 53/96; B01D 53/62; C12M 1/40; C12Q 3/00; C12M 1/12; B01D 53/78; B01D 53/84

(52) U.S. Cl. CPC B01D 53/96; B01D 53/78; B01D 53/62; B01D 53/84; C12Q 3/00; C12M 25/18; C12M 21/18; B01D 2255/804; B01D 2252/204; B01D 2257/504

(57) ABSTRACT

A method for CO₂ capture may include operating a packed reactor comprising a reaction chamber containing packing including immobilized enzymes, by contacting a CO₂ containing gas with a liquid solution in the reaction chamber to produce an ion-loaded solution and a CO₂ depleted gas by an enzymatically catalyzed hydration reaction; monitoring enzyme activity of the immobilized enzymes; at a low enzyme activity threshold (i) stopping operation in the packed reactor, and (ii) replenishing the enzymatic activity by providing an enzyme replenishing solution into the packed reactor to contact the packing and provide a replenishing amount of the immobilized enzymes; and recommencing operation in the packed reactor for CO₂ capture using the replenished immobilized enzymes. A corresponding system may include a packed reactor and an in situ enzyme supply device for supplying active enzyme within the reactor. The enzyme supply device may include spray nozzles with various configurations.

### FIELD OF INVENTION

The present invention generally relates to the field of CO₂ capture or CO₂ absorption. The present invention more particularly relates to the field of enzymatically enhanced CO₂ capture from CO₂ containing gas using a packed reactor and enzyme activity replenishment techniques.

### BACKGROUND

Treatment of CO₂ containing gas has in some cases used the enzyme carbonic anhydrase to enhance the hydration reaction of dissolved CO₂ into bicarbonate and hydrogen ions in an absorption solution. The absorption solution is then treated through precipitation or desorption in order to produce precipitated mineral solids or a relatively pure CO₂ stream for geologic sequestration or reutilization.

Packed reactors having a reaction chamber filled with packing have also been used in the context of CO₂ capture.

In some cases, carbonic anhydrase has been immobilized with respect to packing material in a packed reactor in order to remove CO₂ from an incoming gas.

However, using carbonic anhydrase immobilized to packing in a packed reactor has a number of challenges. For example, over time the carbonic anhydrase present in the packed reactor loses activity notably due to denaturing of the enzyme. While some immobilization materials and techniques can prolong activity levels of the enzyme, over time the enzyme activity reduces and will eventually no longer significantly contribute to the catalysis of the CO₂ hydration reaction. This activity reduction has a number of disadvantages. For instance, consistent process operation over time can be difficult. In addition, removing deactivated packing from a reaction chamber and replacing it with packing that has active enzyme is inefficient, costly and causes unwanted downtime for processing CO₂ containing gas.

### SUMMARY OF INVENTION

The present invention provides techniques for replenishing activity of enzymatic reactors such as packed reactors with enzymatic packing. The present invention also provides techniques for in situ activation of packed reactors.

In some implementations, a method for CO₂ capture includes:
a) operating a packed reactor comprising a reaction chamber containing packing comprising immobilized enzymes, by contacting a CO₂ containing gas with a liquid solution in the reaction chamber to produce an ion-loaded solution and a CO₂ depleted gas by an enzymatically catalyzed hydration reaction;
b) optionally, monitoring enzyme activity of the immobilized enzymes;
c) at a low enzyme activity threshold:
   i) stopping operation in the packed reactor; and
   ii) replenishing the enzymatic activity by providing an enzyme replenishing solution into the packed reactor to contact the packing and provide a replenishing amount of the immobilized enzymes; and
d) recommencing operation in the packed reactor for CO₂ capture using the replenished immobilized enzymes.

Step b) may include monitoring ion concentration in the ion-loaded solution, CO₂ concentration in the CO₂ depleted gas, a gas or liquid concentration in the packed reactor, or an amount of CO₂ released from a downstream desorption reactor.

Step c) i) may include stopping flow of the CO₂ containing gas and/or the liquid solution. Step c) i) may include stopping flow of the liquid solution and drying the packing material.

The enzymes may be entrapped in an immobilization material. The immobilization material may be coated onto the packing. The immobilization material may be spray coated onto the packing. The immobilization material may include polysulfone and/or polysulfone grafted with polyethylene glycol and/or any one or a combination of polymeric materials described in U.S. Pat. No. 7,998,714. The immobilization material may also include micellar polysiloxane material and/or micellar modified polysiloxane materials described in PCT patent application WO 2012/122404 A2. The immobilization material may include chitosan, polyacrylamide and/or alginate. The enzymes may be bonded with an immobilization material to the surface of the packing.

Step ii) may include spraying the enzyme replenishing solution comprising the enzyme and an immobilization material into the packed reactor. The spraying may be performed by nozzles integrated into the packing reactor, by a separate spraying device, and/or by a liquid inlet that provides the liquid solution. The nozzles may be located at a top of the packed reactor, and/or the packed reactor may be composed of several stacks of packing and the nozzles may be at a top location of each stack, and/or located on a side of the packed reactor in one location or arranged along a whole length of the packed reactor.

Step a) may include operating at least two packed reactors in parallel and conducting step c) on only one of the packed reactors at a time.

Step a) may include operating a sufficient number of packed reactors in parallel to be able to continue CO₂ capture on all of the CO₂ containing gas while one of the packed reactors undergoes step c).

The liquid solution may include an absorption compound, wherein the absorption compound includes amines such as primary, secondary and/or tertiary amines; alkanolamines such as primary, secondary and/or tertiary alkanolamines; amino acids such as primary, secondary and/or tertiary amino acids; and/or carbonates and/or aminoether solutions. In some optional aspects, the absorption solution may comprise a chemical compound for enhancing the CO₂ capture process; the solution may further contain at least one compound selected from the following: DEA, DIPA, methyl monoethanolamine (MMEA), TIA, TBEE, HEP, AHPD, hindered diamine (HDA), bis-(tertiarybutylaminoethoxy)-ethane (BTEE), ethoxyethoxyethanoltertiarybutylamine (EEETB), bis-(tertiarybutylaminoethyl)ether, 1,2-bis-(tertiarybutylaminoethoxy)ethane or bis-(2-isopropylaminopropyl)ether, and the like, piperidine, piperazine, derivatives of piperidine or piperazine which are substituted by at least one alkanol group, monoethanolamine (MEA), 2-amino-2-methyl-1-propanol (AMP), 2-(2-aminoethylamino)ethanol (AEE), 2-amino-2-hydroxymethyl-1,3-propanediol (Tris), N-methyldiethanolamine (MDEA), dimethylmonoethanolamine (DMMEA), diethylmonoethanolamine (DEMEA), triisopropanolamine (TIPA), triethanolamine, dialkylether of polyalkylene glycols, dialkylether or dimethylether of polyethylene glycol, amino acids comprising glycine, proline, arginine, histidine, lysine, aspartic acid, glutamic acid, methionine, serine, threonine, glutamine, cysteine, asparagine, valine, leucine, isoleucine, alanine, valine, tyrosine, tryptophan, phenylalanine, and derivatives such as taurine, N,cyclohexyl 1,3-propanediamine, N-secondary butyl glycine, N-methyl N-secondary butyl glycine, diethylglycine, dimethylglycine, sarcosine, methyl taurine, methyl-α-aminopropionic acid, N-(β-ethoxy)taurine, N-(β-aminoethyl)taurine, N-methyl alanine, 6-aminohexanoic acid and potassium or sodium salts of the amino acids; potassium carbonate, sodium carbonate, ammonium carbonate, promoted potassium carbonate solutions and promoted sodium carbonate solutions or promoted ammonium carbonates; or mixtures thereof.

The liquid solution may be a carbonate-based solution, such as potassium carbonate solution, sodium carbonate solution, ammonium carbonate solution, promoted potassium carbonate solutions, promoted sodium carbonate solutions or promoted ammonium carbonates; or mixtures thereof, or promoted with one or more promoter compounds mentioned above.

The enzyme replenishing solution may provide a replenished coating of immobilized enzymes onto the packing. The replenished coating may be provided in a thickness that negligibly increases the size of the packing.

The method may also include, before step c) ii), the step of providing an immobilization material removal fluid into the packed reactor to remove at least some deactivated material.

The method may also include soaking the enzyme replenishing solution for a period of time to substantially coat the packing surface.

The method may also include, before step c) ii), drying the packing using heat, air circulation or circulation of the CO₂ containing gas.

The enzymes and immobilization technique may be provided and the low enzyme activity threshold may be set such that the operation of step a) occurs for a time between about 30 days and about 400 days before requiring enzyme activity replenishment.

Step b) may include continual or periodic monitoring. Step b) may include recognizing a decrease in enzyme activity approaching the low activity threshold and starting preparation of the enzyme replenishing solution to be provided upon reaching the low activity threshold.

Step c) i) may include one or more of the following sub-steps: A) shutting down a flue gas intake in a selected packed reactor, and optionally diverting such gas to another packed reactor or released directly into the atmosphere; B) shutting down the liquid intake, and optionally diverting the liquid to another packed reactor; C) draining the liquid in the shut in packed reactor and optionally thoroughly washing away such liquid; and/or D) optionally adjusting absorption and desorption conditions in accordance with any modified flow rates of the diverted gas and liquid streams.

The method may also include, after step c) ii), allowing a drying time for the immobilized enzymes.

The method may also include performing a co-maintenance activity during step c). The co-maintenance activity comprises cleaning, fouling removal, and/or equipment evaluation checks or replacements.

The method may also include, during step c), venting the CO₂ containing gas.

The method may also include, during step c), utilizing the CO₂ containing gas to enhance immobilization of the enzymes or distribution of the enzymes onto the packing.

In some implementations, a method for CO₂ capture includes:
a) operating a packed reactor comprising a reaction chamber containing packing comprising immobilized enzymes, by contacting a CO₂ containing gas with a liquid solution in the reaction chamber to produce an ion-loaded solution and a CO₂ depleted gas by an enzymatically catalyzed hydration reaction;
b) monitoring enzyme activity of the immobilized enzymes;
c) at a low enzyme activity threshold:
   i) stopping operation in the packed reactor; and
   ii) replenishing the enzymatic activity by removing the packing and replacing with new packing comprising active immobilized enzymes; and
d) recommencing operation in the packed reactor for CO₂ capture using the replenished immobilized enzymes.

The low enzyme activity threshold is based on a lower acceptable performance of the CO₂ capture process.

In some implementations, a method for desorption of an ion-loaded solution includes:
a) operating a desorption reactor comprising packing with immobilized enzymes to produce a regenerated solution and a CO₂ gas by an enzymatically catalyzed dehydration reaction;
b) monitoring enzyme activity of the immobilized enzymes;
c) at a low enzyme activity threshold:
   i) stopping operation in the desorption reactor; and
   ii) replenishing the enzymatic activity by removing the packing and replacing with new packing comprising active immobilized enzymes; and
d) recommencing operation in the desorption reactor for CO₂ desorption using the replenished immobilized enzymes.

In some implementations, a method for CO₂ capture includes:
enzymatically activating a packed reactor comprising a reaction chamber containing packing, by providing an enzyme replenishing solution into the packed reactor to contact the packing and provide a replenishing amount of the immobilized enzymes; and
commencing operation in the packed reactor for CO₂ capture by contacting a CO₂ containing gas with a liquid solution in the reaction chamber to produce an ion-loaded solution and a CO₂ depleted gas by an enzymatically catalyzed hydration reaction.

The method may also include:
providing a surface treatment solution into the reaction chamber to provide a chemical surface treatment to the packing; and
providing one or more solutions, at least one of which comprising a polymeric immobilization material and the enzyme, for immobilizing the enzyme with respect to the packing.

In some implementations, a method for in situ activation of a packed reactor including packing for enzymatic CO₂ capture, includes:
providing at least one enzyme activation solution comprising enzymes into the packed reactor to contact the packing and provide an activating amount of the enzymes immobilized with respect to the packing; and
commencing operation in the packed reactor for CO₂ capture by contacting a CO₂ containing gas with a liquid solution in the reaction chamber to produce an ion-loaded solution and a CO₂ depleted gas by an enzymatically catalyzed hydration reaction.

The method may include:
flowing a first solution (e.g. to provide hydroxyl groups, such as NaOH) through the packed reactor to contact and pre-treat the packing material;
flowing a second solution comprising a functionalizing compound (e.g. APTES) the packed reactor to contact the packing material and produce a functionalized packing;
flowing a third solution comprising a crosslinker (e.g. glutaraldehyde) through the packed reactor to contact the packing material and produce a crosslinker treated packing;
flowing a fourth solution comprising a linker (e.g. polyethylenimine) through the packed reactor to contact the packing material and produce a linker treated packing;
flowing a fifth solution comprising a crosslinker (e.g. glutaraldehyde) through the packed reactor to contact the packing material and produce a pre-treated packing; and
flowing a sixth solution comprising enzyme through the packed reactor to contact the packing material and produce an enzyme activated packing; and
flowing a seventh solution comprising a reducing agent through the packed reactor to contact the enzyme activate packing.

The method may include flowing a cleaning solution (e.g. acid or fluoride solution) through the packed reactor to contact the packing material, prior to the first solution. The method may include the addition of various other or additional solutions to clean, pre-treat, dry, and enzymatically activate the packing, depending on the immobilization technique. Some of the possible solutions and immobilization techniques are described herein.

Methods for replenishment and in situ activation may have a variety of similar optional steps and implementations as described herein.

In another aspect, there may be provided a system for implementing one or more of the methods described above. The system may include a packed reactor comprising a reaction chamber containing packing comprising immobilized enzymes, a gas inlet for receiving a CO₂ containing gas, a liquid solution for receiving a liquid absorption solution into the reaction chamber, a liquid outlet for releasing an ion-loaded solution and a gas outlet for releasing a CO₂ depleted gas. The system may also include an activity monitoring device for monitoring enzyme activity of the immobilized enzymes. The system may also include valves for stopping operation in the packed reactor, by ceasing the flow of the liquid and gas streams entering and exiting the reaction chamber. The system may also include an in situ enzyme supply device for supplying active enzyme to the reaction chamber in order to replenish the enzymatic activity within the reactor. The in situ enzyme supply device may include various spray nozzles, conduits, valves, inlets and outlets, that may include parts of the liquid and gas inlets and outlets used for the operating mode of the packed reactor. One or more of the various features of the system as illustrated in the drawings and described herein may also be included in some embodiments.

### BRIEF DESCRIPTION OF THE DRAWINGS

FIG. 1 is a process flow diagram of an absorption reactor and a desorption reactor.

FIG. 2 is a process flow diagram of an absorption reactor.

FIG. 3 is a process flow diagram of multiple absorption reactors and a desorption reactor.

FIG. 4 is a process flow diagram of an absorption reactor.

FIG. 5 is a process flow diagram of an absorption reactor.

FIGS. 6a to 6c are process flow diagrams of an absorption reactor.

FIGS. 7a to 7d are process flow diagrams of an absorption reactor.

FIG. 8 is a schematic of a packing structure with immobilized carbonic anhydrase.

FIG. 9 is a schematic of a packing structure with immobilized carbonic anhydrase.

FIG. 10 is a schematic of an optional immobilization technique.

### DETAILED DESCRIPTION

The present invention generally relates to enzymatic activity replenishment for packed reactors. In response to enzyme deactivation within the packed reactor, which can occur over time, the activity can be replenished by supplying various fluids in situ to supply new immobilized enzymes and thereby increase the enzymatic activity in the packed reactor.

Optional Aspects of CO₂ Capture System and Methods with Activity Replenishment

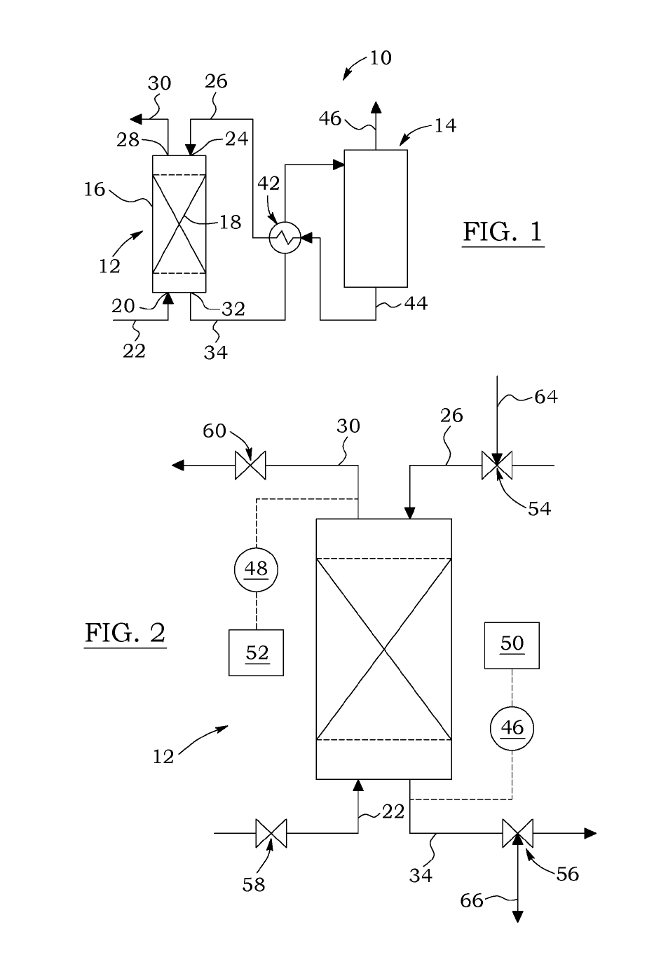

Referring to FIG. 1, the CO₂ capture system 10 may include an absorption reactor 12 and a desorption reactor 14. The absorption reactor is preferably a packed reactor having a reaction chamber 16 that is filled with packing 18. The absorption reactor has a gas inlet 20 for providing a CO₂ containing gas 22, a liquid inlet 24 for providing an absorption solution 26, a gas outlet 28 for releasing a treated gas 30 depleted in CO₂ and a liquid outlet 32 for releasing an ion loaded solution 34.

The CO₂ containing gas 22 enters the reaction chamber and contacts the absorption solution 26. The CO₂ dissolves into the absorption solution where it is chemically transformed into hydrogen and bicarbonate ions by hydration reaction catalysed by carbonic anhydrase present in the reaction chamber.

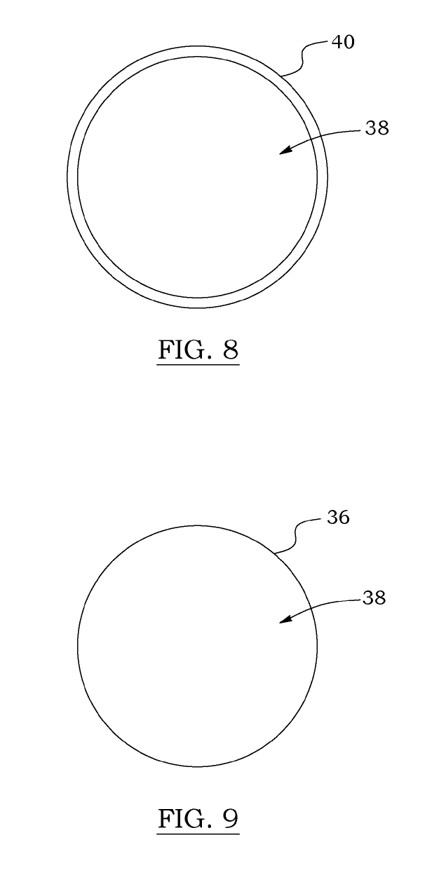

The carbonic anhydrase may be immobilized with respect to the packing material. Referring to FIG. 9, the carbonic anhydrase 36 may be immobilized directly onto the packing structures 38. Referring to FIG. 8, the carbonic anhydrase may be immobilized with respect to an immobilization material 40 that is coated or otherwise bonded to the packing structures 38. The immobilization technique may include covalent bonding, entrapment, encapsulation, or another technique.

Referring back to FIG. 1, the ion loaded solution 34 may be supplied to the desorption reactor 14. The ion loaded solution 34 may be heated in a heat exchanger 42 before desorption. The desorption reactor 14 produces a regenerated solution 44 and a CO₂ gas 46. The regenerated solution 44 is then provided back into the absorption reactor as at least part of the absorption solution 26. The regenerated solution 44 may pass through the heat exchanger 42 for heating the ion loaded solution 34.

In one aspect of the invention, there are methods for replenishing enzyme activity in the absorption reactor, such as the one illustrated and used in the system 10 of FIG. 1.

One activity replenishment method may include replenishing the enzymatic activity by providing an enzyme replenishing solution into the packed reactor to contact the packing and provide a replenishing amount of the immobilized enzymes. In one aspect, there is an overall process for CO₂ capture including the following steps:
a) operating a packed reactor comprising a reaction chamber containing packing comprising immobilized enzymes, by contacting a CO₂ containing gas with a liquid solution in the reaction chamber to produce an ion-loaded solution and a CO₂ depleted gas by an enzymatically catalyzed hydration reaction;
b) monitoring enzyme activity of the immobilized enzymes;
c) at a low enzyme activity threshold:
   i) stopping operation in the packed reactor; and
   ii) replenishing the enzymatic activity by providing an enzyme replenishing solution into the packed reactor to contact the packing and provide a replenishing amount of the immobilized enzymes; and
d) recommencing operation in the packed reactor for CO₂ capture using the replenished immobilized enzymes.

In some aspects, step b) includes monitoring ion concentration in the ion loaded solution, CO₂ concentration in the CO₂ depleted gas, a gas or liquid concentration in the packed reactor, or an amount of CO₂ released from a downstream desorption reactor.

Referring to FIG. 2, the absorption reactor 12 may include a liquid measurement device 46 and/or a gas measurement device 48 for measuring one or more properties of the treated gas 30 or the ion loaded solution 34. The system may include a first and/or second controllers 50, 52 for controlling operational parameters of the absorption process and/or a replenishment protocol. Step b) may include continual or periodic monitoring.

Still referring to FIG. 2, the absorption reactor 12 may include various valves for adjusting or stopping the flow of gas or liquid entering and exiting the absorption reactor 12. There may be a liquid inlet valve 54, a liquid outlet valve 56, a gas inlet valve 58 and a gas outlet valve 60.

Step c) i) may include stopping flow of the CO₂ containing gas and/or the liquid solution. This may be done by closing valves 54, 56, 58 and 60. Step c) i) may include stopping flow of the liquid solution and then drying the packing material, which may be done by various means including continuing to inject the gas 22 and thus the gas valves 58, 60 would remain open for a certain amount of time. Step c) i) may include the following: A) shutting down a flue gas intake in a selected packed reactor, this gas could be diverted to another absorber or released directly into the atmosphere. If continuous operation is not required, the plant could be shut down. B) The liquid absorbing solution intake may be shut down. If the system comprises only one absorber, the entire CO₂ capture unit should be shut down. If other absorber(s) is/are present, liquid flow rate in the other absorber(s) should be adjusted and desorbing conditions should be adapted. C) The liquid phase in the stopped absorber may be drained. If this liquid is incompatible with the enzyme regeneration process, it should be thoroughly washed away.

Before step c) ii), the process may include drying the packing using heat, air circulation and/or circulation of the CO₂ containing gas.

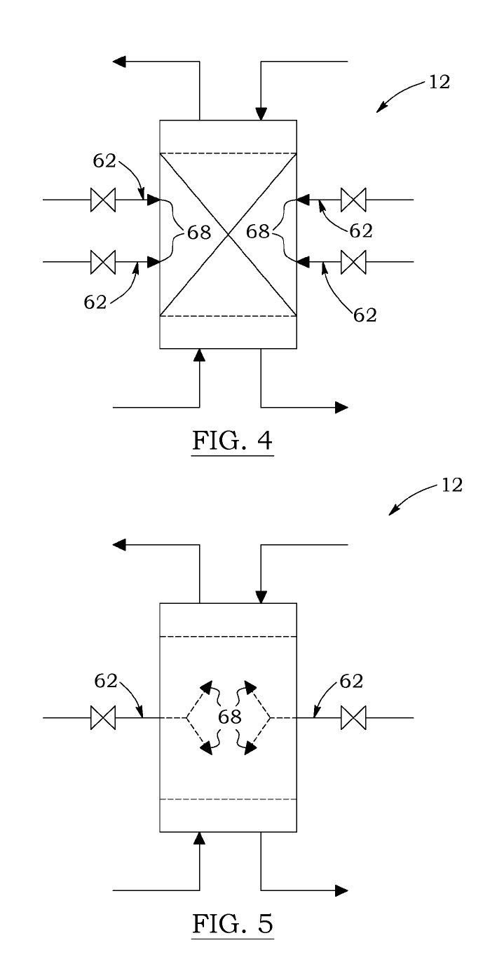

Step c) ii) may include spraying the enzyme replenishing solution comprising the enzyme and an immobilization material into the packed absorption reactor 12. Referring to FIG. 4, the spraying may be performed by spraying inlets 62. The spraying may be done using nozzles integrated into the packing reactor, by a separate spraying device, and/or by a liquid inlet (24 in FIG. 1) that provides the absorption solution 26. FIG. 2 shows that the liquid inlet valve 54 may be a three way valve so that the replenishing solution 64 may be sprayed into the reactor. The liquid outlet valve 56 may also be a three way valve for releasing the spent replenishing fluid 66 that drains through the reactor 12.

The spraying inlets 62 may include nozzles 68 that are provided within the reaction chamber (as in FIG. 5) or at the perimeter of the reaction chamber (as in FIG. 4). The nozzles may be provided on the sides or top of the reactor 12.

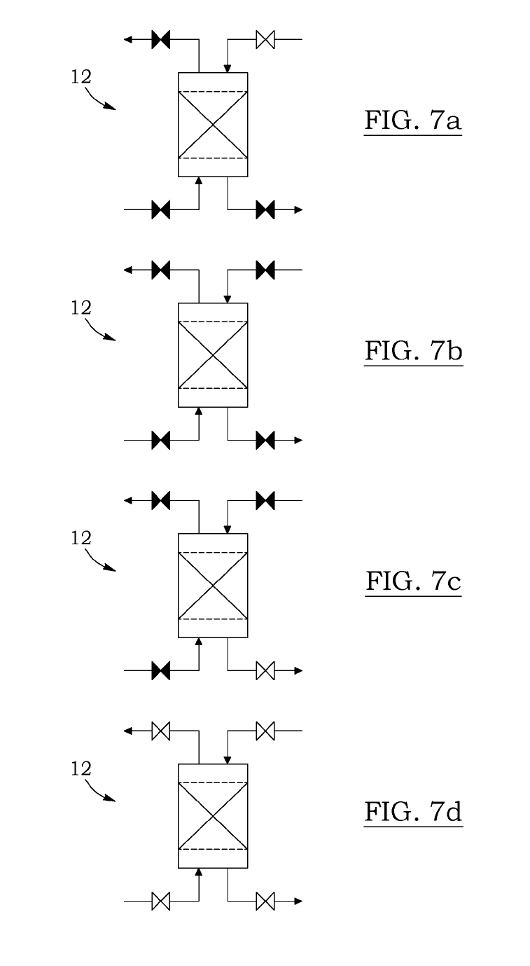

FIGS. 7a to 7d show one valve protocol that may be used. In FIG. 7a, the replenishing solution is supplied to the reaction chamber via the liquid inlet. As in FIG. 7b, there may be a contact or soaking period during which the replenishing solution is allowed to contact the packing material, depending on the immobilization technique by which the enzymes are provided on the packing. For large scale applications for which the absorption column(s) and the reactor volumes would be large, it may be preferred to contact the replenishing solutions with the packing material rather than filing the absorption column with the solution for soaking. In such a case, the contacting step would require valve operation to allow the replenishing solution to flow through the reactor rather than fill it. In addition, if soaking is performed, the absorption column construction should be sufficient to support the extra weight of the replenishing solution during the soaking period. The contacting or soaking of the enzyme replenishing solution may be done for a period of time to substantially coat the packing surface. In the case of contacting without soaking, the replenishing solution may be re-circulated through the packed reactor for a sufficient time to ensure the packing material is re-activated. In FIG. 7c, any remaining spent replenishing liquid may be withdrawn through the bottom liquid outlet line. In FIG. 7d, the process is re-commenced and the gas and liquid lines are re-opened.

The activity replenishment method may include several optional steps, such as the following:

(I) Flowing a removal solution through the packing that will enable to remove partially or totally the enzyme previously present at the surface of the packing. This removal solution may contain an acid, a base, a salt or another compound or mixture that would remove or destroy the coating at the surface of the packing.

(II) Flowing a surface preparation solution for regeneration of the chemical groups at the surface of the packing may be desirable in the case that a certain surface chemistry is required or desirable for the immobilization of the enzymes with respect to the packing. Chemical groups at the surface of the packing may act as anchor points for the immobilization of the enzymes in subsequent steps. This treatment may produce a surface treated packing material. One or more surface treatments may be performed to provide a given immobilization.

(III) Flowing at least one solution, or multiple solutions in a given sequence, containing chemicals (including enzyme) responsible for different reactions required to immobilize the enzyme at the surface of the packing, which will react with the surface of the packing. These solutions may contain only one compound, or a mixture of compounds. The compounds may include chemicals such as crosslinkers (glutaraldehyde, dextran polyaldehyde), linkers (polyethyleneimine, ethylene diamine, polyamines), buffers (phosphate, carbonates, Tris, etc.), polymer (chitosan, polyacrylamide, polysulfone, polysulfone grafted with polyethylene glycol, and/or any polymeric immobilization material described in U.S. Pat. No. 7,998,714).

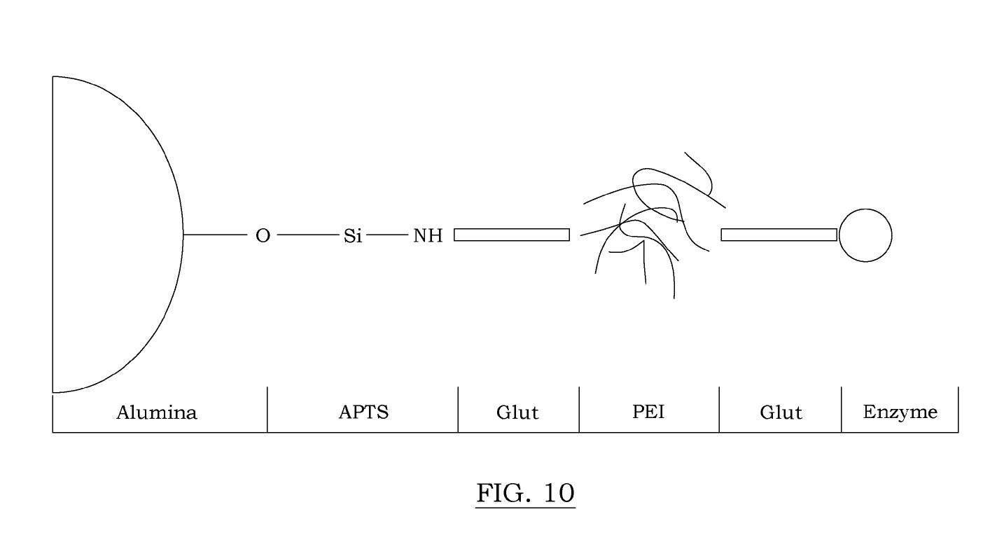

In one optional scenario, carbonic anhydrase may be immobilized with respect to alumina or ceramic packing. Referring to FIG. 10, for example, the immobilization may include chemical link between the enzyme and the alumina packing via APTES, glutaraldehyde and PEI. In the event that the alumina packing previously had immobilization for a CO₂ capture operation in a packed reactor, there may be a step of removing the coating, for example using strong acid or fluoride compound like tetra-n-butylammonium fluoride. The removal solution may be flowed through the packed reactor for in situ removal of the coating. The hydroxyl group at the surface of the packing may then be regenerated using a treatment with NaOH solution. This solution may be flowed through the packed reactor and may optionally be collected for re-use. The packing may then be functionalized by contacting with APTES (3-aminopropyltriethoxysilane) in toluene solution at 80° C., for example. A heated toluene based solution including APTES may be flowed through the packed reactor, and it may optionally be collected for re-use. The packing may then be washed, for example with methanol and water. This solution may also be collected. Then, a glutaraldehyde (crosslinker) may be added using glutaraldehyde in a carbonate buffer. The packing may then be washed with water. PEI (polyethylenimine, a linker) may then be added using PEI in a carbonate buffer. The packing may then be washed again. Then another glutaraldehyde may be added using glutaraldehyde in a carbonate buffer. The packing may then be washed again with water. The enzyme may then be added as a solution with carbonate buffer and carbonic anhydrase The packing may then be washed. The imine bonds may then be reduced, by adding a reducing agent, such as NaBH₃CN.

In another optional scenario, carbonic anhydrase may be adsorbed on a porous packing. If the enzymatic packing has already been in operation in the packed reactor, the enzyme may be stripped from the support using a base, an acid, an organic solution, a concentrated saline solution, or a combination of such treatments (e.g. in sequence). Once the enzyme has been stripped, the packing may be washed to remove the stripping solution(s). A solution containing the enzyme is then applied to the packing, for example using a variety of solution application techniques such as spraying. After application of the enzyme solution, excess enzyme may be washed away.

In another optional scenario, carbonic anhydrase may be embedded into a polymeric coating that is coated over packing. If necessary, the enzyme-polymer may be stripped from the packing using a base, an acid, an organic solution, a concentrated saline solution or a combination of such treatments (e.g. in sequence) or any other compound(s) that can break, dissolve and/or remove the coating. The packing may then be washed to remove the stripping solution(s). A solution containing the enzyme-polymer mixture may then be applied to the packing, for example using a variety of solution application techniques such as spraying. After application of the enzyme solution, excess liquid may be drained and the coating may be dried, for example using air or flue gas.

In another scenario, carbonic anhydrase may be covalently bonded to the packing or to an immobilization material that is provided as a coating of the packing. The packing may be chemically pre-treated to provide one or more appropriate functional groups for attachment of the enzyme. For example, the methods disclosed by Zhang et al.'s article entitled Catalytic behavior of carbonic anhydrase enzyme immobilized onto nonporous silica nanoparticles for enhancing CO₂ absorption into a carbonate solution (International Journal of Greenhouse Gas Control 13(2013) 17-25), for synthesizing enzymatic nanoparticles maybe adapted with similar chemistry for providing immobilized carbonic anhydrase on a ceramic packing material. The pre-treatment methods described by Zhang et al. may also be used in conjunction with the present process for preparing packing material for immobilization of the enzyme. Other methods of bonding enzyme to packing or coating materials may also be used.

As may be understood from the above examples, there may be several solution addition steps in order to clean, surface treat, functionalise and wash the packing in order to activate the packing with carbonic anhydrase. In addition, between each or some of the successive steps of (I), (II) and (III), there may be one or more washing steps to remove excess chemicals that could interfere with subsequent steps. The washing may be done with water or another fluid depending on the previous treatment and the subsequent treatment requirements. There may be also some steps where a gas is flowed through the packing to let the immobilization material or chemicals dry.

This method may be used by contacting the solutions using spraying and allowing the solutions to flow through the packing, or by filling the reactor and using a soaking technique.

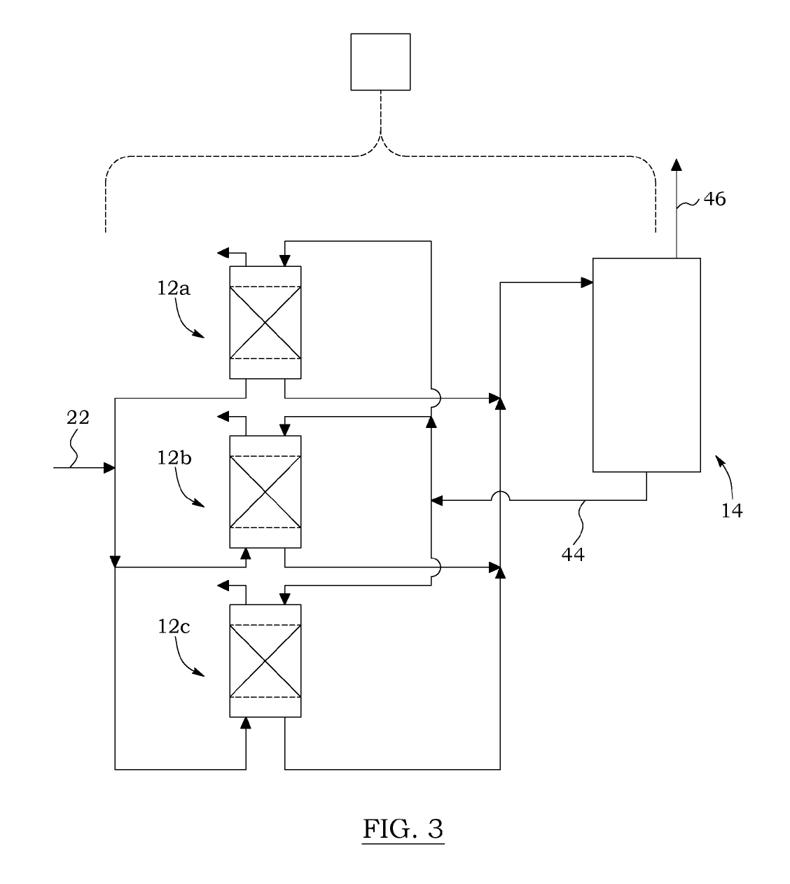

Referring now to FIG. 3, step a) may include operating at least two packed reactors in parallel and conducting step c) on only one of the packed reactors at a time. FIG. 3 illustrates three absorption reactors 12a, 12b, 12c, each of which may be operated and constructed as the reactor 12 in FIG. 1, 2 or 4 to 7d. Step a) may include operating a sufficient number of packed reactors 12 in parallel to be able to continue CO₂ capture on all of the CO₂ containing gas 22 while one of the packed reactors undergoes step c).

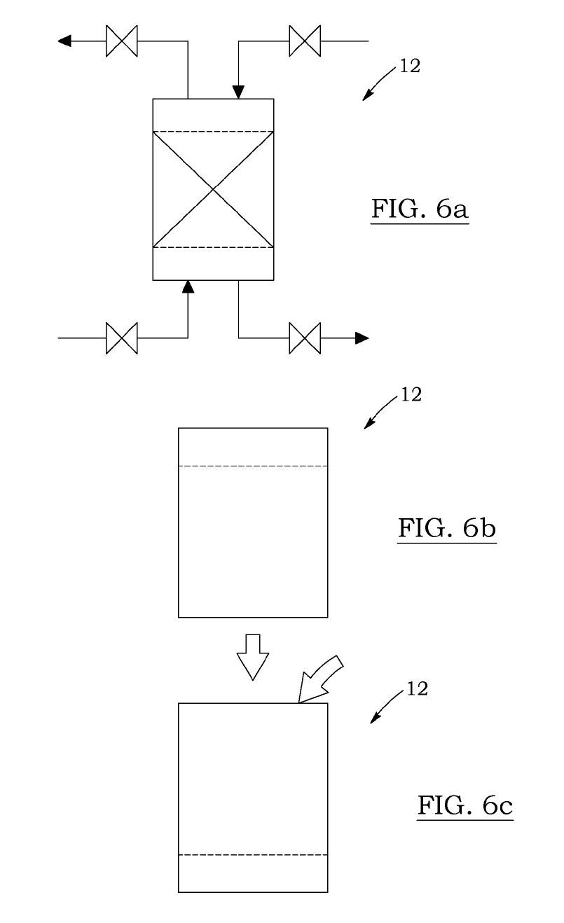

In some aspects, the packing material may be removed from one of the absorption reactors 12, as generally illustrated in FIGS. 6a to 6c. The inlets are closed and the bottom retention grill is removed to empty the packing material from the bottom as in FIG. 6b. New active packing material or the removed packing material that has been re-activated with enzyme can then be put back into the reaction chamber from the top as in FIG. 6c.

The packing may be introduced in the column as different sections of fixed volume. The packing may be removed from the packed column one section at a time to enable easy handling of the packing. If the column has multiple sections including packing material, the sections may be replenished together or individually, depending on the nozzle, valve and piping configurations that are provided.

In some aspects, the enzyme replenishing solution may provide a replenished coating of immobilized enzymes onto the packing. The coating may include an enzyme immobilization material that enables entrapment of the enzymes within pores of the immobilization material.

In some aspects, the replenished coating may be provided in a thickness that negligibly increases the size of the packing. If immobilisation material is sprayed periodically onto a previous inactivated layer, then the size of the packing material may increase. The replenishing solution and the spraying method may be controlled to minimize the thickness of each subsequent coating.

In some aspects, the process may also include, before step c) ii), the step of providing an immobilization material removal fluid into the packed reactor to remove at least some deactivated material. This may be done for each replenishment protocol, or only when desired, e.g. when several coatings have increased the size of the packing beyond a desirable level or when the coating is too thick to have layered coatings.

Regarding step b), it may also include recognizing a decrease in enzyme activity approaching the low activity threshold and starting preparation of the enzyme replenishing solution to be provided upon reaching the low activity threshold.

After step c) ii), there may be a step of allowing a drying time for the immobilized enzymes.

The process may also include performing a co-maintenance activity during step c). The co-maintenance activity may be cleaning, fouling removal, and/or equipment evaluation checks or replacements.

In another aspect, there is a method for CO₂ capture, including:
a) operating a packed reactor comprising a reaction chamber containing packing comprising immobilized enzymes, by contacting a CO₂ containing gas with a liquid solution in the reaction chamber to produce an ion-loaded solution and a CO₂ depleted gas by an enzymatically catalyzed hydration reaction;
b) monitoring enzyme activity of the immobilized enzymes;
c) at a low enzyme activity threshold:
   i) stopping operation in the packed reactor; and
   ii) replenishing the enzymatic activity by removing the packing and replacing with new packing comprising active immobilized enzymes; and
d) recommencing operation in the packed reactor for CO₂ capture using the replenished immobilized enzymes.

In another aspect, there is a method for desorption of an ion-loaded solution, comprising:
a) operating a desorption reactor comprising packing with immobilized enzymes to produce a regenerated solution and a CO₂ gas by an enzymatically catalyzed dehydration reaction;
b) monitoring enzyme activity of the immobilized enzymes;
c) at a low enzyme activity threshold:
   i) stopping operation in the desorption reactor; and
   ii) replenishing the enzymatic activity by removing the packing and replacing with new packing comprising active immobilized enzymes; and
d) recommencing operation in the desorption reactor for CO₂ desorption using the replenished immobilized enzymes.

The techniques described for the absorption reactor 12 may be provided and adapted as needed for a packed desorption reactor 14.

Regarding the low enzyme activity threshold, it will depend in the particular operating conditions of the CO₂ capture process. In some aspects, the low enzyme activity threshold may correspond to a minimum acceptable level for CO₂ capture for the absorption unit corresponding to a minimum performance, which may be a minimum performance required to meet an environmental legislation requirement. For example, if the minimum CO₂ removal level is 90%, and the initial performance of the CO₂ capture unit is about 95%, then as the result of the immobilized enzyme loss of activity, the global CO₂ capture performance will decrease until it reaches the low threshold performance of 90%. When this value is reached the procedure for replenishing enzyme activity may be initiated. It should be noted that the low enzyme activity threshold may be defined in other ways; for example it may be defined as the activity below which the CO₂ capture process is below economic or technical performance requirements for the given CO₂ capture operation. In addition, if the CO₂ capture operation is coupled to another industrial operation, such that a product of the CO₂ capture operation (e.g. bicarbonate loaded solution, CO₂ gas, etc.) is used in a certain minimum amount in the industrial operation, then the low enzyme activity threshold may be activity required to produce enough of the product for the industrial operation.

It should also be noted that the steps c) i), c) ii) and d) may be adapted for a method of activating a packed reactor that did not previously have enzymes immobilized on its packing. This may be useful for retrofitting applications where a packed reactor may have been implemented for a CO₂ capture operation and the performance of the operation is to be enhanced by the addition of enzymes to the packing material. In this case, since removing and re-installing the packing may be expensive and challenging, an enzyme activation protocol may be implemented for providing immobilized enzyme on the packing within the reactor. The steps (I), (II) and (III) described above may be used for this activation method, although step (I) in particular could be avoided if the packing was not previously coated.

The documents referred to herein, such as U.S. application Ser. No. 12/984,852, U.S. Pat. No. 7,998,714, Zhang et al., and PCT patent application WO 2012/122404 A2 are hereby incorporated herein by reference. Many different immobilization techniques and solutions for immobilizing carbonic anhydrase for replenishment and/or in situ activation of packed reactors, including various combinations of aspects described herein, may be used, some of which may be adapted from the descriptions in such documents.

It should also be noted that various modifications may be made to the techniques described herein, such as the use of different types of solutions, enzymes, flue gases, packing material compositions and forms, and so on.

### Example Scenarios

### Example Scenario 1: Activity Replenishment of Enzyme Immobilized on the Surface of Random or Structured Ceramic or Steel Packing in a Packed Column—Addition of Fresh Enzyme

Enzyme immobilization directly on the surface of the packing may involve the following steps:
(i) Etching of the packing surface to introduce surface hydroxide groups;
(ii) Surface functionalization using isocyanate groups, or alkoxy-silane groups or allyl groups;
(iii) Reacting a linker with the functional groups added in step 2; the linker may provide an anchor for direct enzyme fixation (for example glutaraldehyde); and
(iv) Enzyme fixation.

In a CO₂ capture process, when the process performance decreases because of the enzyme deactivation, activity replenishment may take place. One strategy is to immobilize fresh enzyme to the surface of the packing containing the previous enzyme, which may be done as follows:
1. After stopping the CO₂ absorption operations, rinse packing with water until pH is close to neutral.
2. Prepare a buffer solution containing a linker, such as glutaraldehyde, and feed the solution to the packed column. The solution may be sprayed from top, or side of the packing. The solution may be recycled to the packed column to provide a sufficient reaction time of the linker with packing. The linker reacts with amino groups present at the surface of the old enzyme and/or other reactants used during first immobilization.
3. When step 2 is completed, the packing may be washed, e.g. with water or buffer solution, to remove excess linker.
4. Then a buffer solution containing the enzyme carbonic anhydrase is prepared and fed to the packed column. The buffer composition (i.e. buffer species), buffer concentration and enzyme concentration are set to obtain optimal enzyme immobilization giving higher fixation level and highest activity level. As was the case for step 2, the solution can be recycled to enable a sufficient reaction time for the enzyme to attach to the packing. Additional steps can be performed such as a chemical reduction step, if chemical stabilization of the chemical bonds between the enzyme and the linker is desired. It should be noted that in some cases the enzyme may be chemically modified prior to its immobilization to facilitate attachment of the enzyme to the packing and/or the coating. Various chemical modifications may be performed depending on the type and method of immobilization. For example, chemical modification may include providing free thiol groups that can react with maleimides, iodoacetamides, pyridyl disulfides, or vinyl sulfones. It is also possible to modify other groups, such as hydroxyls, amino, carboxylic and sulfhydryl, prior to immobilizing the enzyme with respect to the packing. It should also be noted that this chemical modification pre-treatment may be done in various other example scenarios described herein.
5. The excess solution is drained and packing is rinsed with the CO₂ absorption solution to be used. The material is then ready for servicing.

Note that for the different steps, the solutions may be sprayed from top, or side of the packed column.

### Example Scenario 2: Activity Replenishment of Enzyme Immobilized on the Surface of Random or Structured Ceramic or Steel Packing in a Packed Column—Old Enzyme is Removed and Replaced with Fresh Enzyme

Enzyme immobilization directly on the surface of the packing could involve the following steps:
(i) Etching of the packing surface to introduce surface hydroxide groups;
(ii) Surface functionalization using isocyanate groups, or alkoxy-silane groups or allyl groups;
(iii) Reacting a linker with the functional groups added in step 2; the linker may provide an anchor for direct enzyme fixation (for example glutaraldehyde); and
(iv) Enzyme fixation.

Another strategy for activity replenishment is to remove the old immobilization material and replace it with fresh material. The steps may be as follows:
1. After stopping the CO₂ absorption operations, rinse packing with water until pH is close to neutral.
2. Prepare a strong base solution comprising compounds such as NaOH or KOH (pH over 12) and feed it into the packed column. Contact time, concentration and temperature may be adjusted to remove the functional groups added at step (ii) mentioned above. This step can also be performed using a strong acid solution with a pH below 2 for example. Strong acid solutions may contain HF, HNO₃ and/or hydrogen peroxide.
3. The packing is then rinsed with water to remove the strong base or strong acid solution and the immobilization chemicals leached off the packing. Water wash is performed to reach a neutral pH.
4. Prior to the next step, the packing is preferably dried to remove excess water. Air or the CO₂ containing gas may be used for the operation.
5. The surface of the packing is then chemically modified to add isocyanate groups, or alkoxy-silane groups or allyl groups. These groups will react with functional groups found in the coating and make the coating strongly attached to the packing surface.
6. If step 5 is performed in an aqueous solution, the packing is then rinsed with water to remove the excess of reactants used in step 5. Water wash is performed to reach a neutral pH. In the event that step 5 is performed in an organic solvent, the same solvent is used for rinsing.
7. Prepare a buffer solution containing a linker such as glutaraldehyde or a molecule containing an aldehyde group or an epoxy group, and feed the solution into the packed column. The solution could be sprayed from top, or side of the column. The solution could be released and recycled back into the packed column to provide a sufficient reaction time of the linker with the packing. The linker will react with amino groups or other functional groups added at Step 5.
8. When step 7 is completed, the packing is washed to remove excess linker still present. This washing step might be performed using water or buffer solution used at step 9.
9. Then a buffer solution containing the enzyme carbonic anhydrase is prepared and fed to the packed column. As was the case for step 2, the solution can be recycled to enable a sufficient reaction time for the enzyme to attach to the packing. Additional steps can be performed such as a chemical reduction step, if chemical stabilization of the chemical bonds between the enzyme and the linker is desired.
10. The excess solution is drained and packing is rinsed with the CO₂ absorption solution to be used. The material is then ready for servicing.

Note that for the different steps, the solution could be sprayed from the top, or side of the packed column.

### Example 3: Activity Replenishment of Enzyme Immobilized Inside a Porous Coating Fixed to the Surface of a Random or Structured Ceramic or Steel Packing in a Packed Column—Addition of Fresh Enzyme

Enzyme immobilization in a porous coating on the surface of a packing could include the following steps:
(i) Etching of the packing surface to introduce surface hydroxide groups;
(ii) Surface functionalization using isocyanate groups, or alkoxy-silane groups or allyl groups;
(iii) Prepare a solution containing the polymers and the enzyme that will form the coating;
(iv) Contact the packing with the polymer-enzyme solution to enable the polymer-enzyme solution to coat all the surface of the packing;
(v) Place the packing material on a grid, support, enable excess of solution to be removed and then the coating to dry;
(vi) Expose the coated packing to higher temperature (e.g. 40-80° C.) for few hours to complete curing of the coated packing. Temperature should also be selected in such a way that the enzyme in the coating is not denatured.

Another strategy for activity replenishment is to fix fresh enzyme directly onto the surface of the coating, which may be done as follows:
1. After stopping the CO₂ absorption operations, rinse the packing with water until pH is close to neutral. This operation can also be done using an organic solvent. It depends on the solution required for step 2.
2. Prepare a solution containing a linker. The solution composition is selected to be compatible with chemical nature of the linker. It may consist of an aqueous buffer, or an organic solvent. Linker may be selected for its ability to chemically react with the polysiloxanes or modified polysiloxanes and/or the enzyme. The linker is selected considering the functional groups available at the surface of the coating. For example, if amino groups are present, glutaraldehyde can be used as a linker. Linker with epoxy functional group can also be used. The linker maybe an aminated silane or an epoxy silane, for example.
3. Prepare a buffer solution containing the linker, and feed the solution to the packed column. The solution may be sprayed from the top, or the side of the packed column. The solution could be recycled to provide a sufficient reaction time of the linker with the packing.
4. When step 3 is completed, the packing is washed with water or buffer solution to remove excess linker still present.
5. Then a buffer solution containing the enzyme carbonic anhydrase is prepared and fed to the packed column. As was the case for step 2, the solution can be recycled to enable a sufficient reaction time for the enzyme to attach to the packing. Additional steps can be performed, such as a chemical reduction step, if chemical stabilization of the chemical bonds between the enzyme and the linker is desired.
6. The excess solution is drained and packing is rinsed with the CO₂ absorption solution to be used. The material is then ready for servicing.

### Example 4: Activity Replenishment of Enzyme Immobilized Inside a Porous Coating Fixed to the Surface of a Random or Structured Ceramic or Steel Packing in a Packed Column—Removing the Old Coating and Replacement by a New Coating with Fresh Enzyme

Enzyme immobilization in a coating on the surface of the packing could involve the following steps:
(i) Etching of the packing surface to introduce surface hydroxide groups;
(ii) Surface functionalization using isocyanate groups, or alkoxy-silane groups or allyl groups;
(iii) Prepare a solution containing the polymers and the enzyme that will form the coating;
(iv) Contact the packing with the polymer-enzyme solution to enable the polymer-enzyme solution to coat all the surface of the packing;
(v) Place the packing material on a grid, support, enable excess of solution to be removed and then the coating to dry;
(vi) Expose the coated packing to higher temperature (e.g. 40-80° C.) for few hours to complete curing of the coated packing. Temperature should also be selected in such a way that the enzyme in the coating is not denatured.

A further enzyme replenishment strategy is to remove the old coating and adding a new coating made of fresh enzyme, which may be done as follows:
1. After stopping the CO₂ absorption operations, rinse the packing with water until pH is close to neutral.
2. Prepare a strong base solution comprising compounds such as NaOH or KOH (pH over 12) and feed it into the packed column. Contact time, concentration and temperature may be adjusted to remove the functional groups added at step (ii) above. This step can also be performed using a strong acid solution with a pH below 2. Strong acid solutions may contain HF, HNO₃ and/or hydrogen peroxide.
3. The packing is then rinsed with water to remove the strong base or strong acid solution and the immobilization chemicals are leached off the packing. Water wash is performed to reach a neutral pH.
4. Prior to the next step, the packing is preferably dried to remove excess water. Air or the CO₂ containing gas may be used for the drying operation.
5. The surface of the packing is then chemically modified to add isocyanate groups, or alkoxy-silane groups or allyl groups. These groups can react with functional groups found in the coating and make the coating strongly attached to the packing surface.
6. If step 5 is performed in an aqueous solution, the packing is then rinsed with water to remove the excess of reactants used in step 5. Water wash is performed to reach a neutral pH. In the case that step 5 is performed in an organic solvent, the same or similar solvent may be used for rinsing.
7. If water is used in step 6, the packing is preferably dried prior to next step. Air or the CO₂ containing gas might be used for this purpose.
8. Prepare a polymer-enzyme mixture. The polymer may include, for example, a mixture of polysiloxane and/or modified polysiloxanes. The mixture may also contain catalyst(s) or chemicals for the crosslinking reaction between the polysiloxanes and/or modified polysiloxanes and the enzyme. Prior to its use for preparing the mixture, the enzyme may be chemically modified in such a way that the enzyme can chemically react with the polymer and then be physically and chemically immobilized to the coating. Some possible chemical modifications are described above in another example scenario. It should also be noted that the chemical modifications and/or preparation of polymer-enzyme mixture and its application on the packing (as per step 5) may use various different techniques, for example techniques described in U.S. patent application Ser. No. 12/984,852; PCT patent application WO 2012/122404 A2; and U.S. Pat. No. 7,998,714. The polymer-enzyme mixture may be formulated and applied so as to form a polymeric micellar or inverted micellar immobilization material coating the packing in the column. The immobilization material may be or include polysulfones, polycarbonates, poly(vinylbenzyl chlorides) and/or polysiloxanes. The mixture may include a cross-linking agent to enable cross-linking of the polymer to provide a cross-linked polymeric immobilization material. The immobilization material may also include other components, such as a metal catalyst and the like. It should be noted that the features described above are not limited to this example scenario but may also be used in various other methods and/or example scenarios described herein.
9. Feed the polymer-enzyme solution to the packed column. The solution may be sprayed from the top, or the side of the packed column. The solution may be recycled back into the packed column to provide a sufficient reaction time of the linker with packing.
10. Air or a CO₂ containing gas is blown through the packing bed, to enable removing excess polymer-enzyme solution to obtain a thin coating on the packing. The gas flow (or air flow) could also be heated to temperatures ranging from 40 to 80° C. to facilitate curing of the coating. The heated gas could be at least partly from hot CO₂ containing gas, such as hot exhaust gases and the like, which are provided at an appropriate temperature.
11. Then the packing with the new coating is washed and conditioned with the CO₂ absorption solution.
12. After the conditioning step, the packing is ready for CO₂ capture operations.

### Example 5: Activity Replenishment of Enzyme Immobilized Inside a Porous Coating Fixed to the Surface of a Random or Structured Ceramic or Steel Packing in a Packed Column—Adding a New Coating on the Top of the Old Coating

Enzyme immobilization directly on the surface of the packing could involve the following steps:
(i) Etching of the packing surface to introduce surface hydroxide groups;
(ii) Surface functionalization using isocyanate groups, or alkoxy-silane groups or allyl groups;
(iii) Prepare a solution containing the polymers and the enzyme that will form the coating;
(iv) Contact the packing with the polymer-enzyme solution to enable the polymer-enzyme solution to coat all the surface of the packing;
(v) Place the packing material on a grid, support, enable excess of solution to be removed and then the coating to dry;
(vi) Expose the coated packing to higher temperature (e.g. 40-80° C.) for few hours to complete curing of the coated packing. Temperature should also be selected in such a way that the enzyme in the coating is not denatured.

Yet a further enzyme replenishment strategy is to add a new coating over the old coating, which may be done as follows:
1. After stopping the CO₂ absorption operations, rinse the packing with water until pH is close to neutral.
2. Prior to the next step, the packing is preferably dried to remove excess water. Air or the CO₂ containing gas may be used for the operation.
3. The surface of the old coating is then chemically modified to add isocyanate groups or alkoxy-silane groups or allyl groups. Depending on the functional group to be added, the chemical reactions may take place in an aqueous buffer or an organic solvent. These groups will react with functional groups found in the coating and make the new coating to strongly attach to the surface of the old packing.
4. The packing is then rinsed with the solution used in step 3 to remove the excess of reactants used in step 2.
5. If an aqueous solution or water is used in step 4, packing is preferably dried prior to next step. Air or the CO₂ containing gas can be used for this purpose.
6. Prepare a polymer-enzyme mixture. The polymer may include, for example, a mixture of polysiloxane and/or modified polysiloxanes. The mixture may also contain catalyst(s) or chemicals for the crosslinking reaction between the polysiloxanes and/or modified polysiloxanes and the enzyme. Prior to its use for preparing the mixture, the enzyme may be chemically modified in such a way that the enzyme can chemically react with the polymer and then be physically and chemically immobilized to the coating
7. Feed the polymer-enzyme solution to the packed column. The solution may be sprayed from the top, or the side of the packed column. The solution may be recycled back into the packed column to provide a sufficient reaction time of the linker with packing.
8. Air or a CO₂ containing gas is blown through the packing bed, to enable removing excess polymer-enzyme solution to obtain a thin coating on the packing. The gas flow (or air flow) could also be heated to temperatures ranging from 40 to 80° C. to facilitate curing of the coating. The heated gas could be at least partly from hot CO₂ containing gas, such as hot exhaust gases and the like, which are provided at an appropriate temperature.
9. Then the packing with the new coating is washed and conditioned with the CO₂ absorption solution.
10. After the conditioning step, the packing is ready for CO₂ capture operations.

The documents referred to herein are incorporated herein by reference in their entirety.

What is claimed is:

1-45. (canceled)

46. A method for desorption of an ion-loaded solution comprising hydrogen and bicarbonate ions, the method comprising:
supplying ion-loaded solution into a desorption reactor comprising packing with immobilized enzymes provided on the packing;
operating the desorption reactor to produce a regenerated solution and a CO₂ gas by an enzymatically catalyzed dehydration reaction;
at a low enzyme activity threshold:
stopping operation in the desorption reactor; and
subjecting the desorption reactor to in situ activity replenishment, comprising:
removing the ion-loaded solution from the desorption reactor;
providing at least one enzyme activation solution comprising enzymes into the packed reactor to contact the packing;
coating the enzyme activation solution onto the packing to form a wet coating;
curing the wet coating to provide an activating amount of the enzymes immobilized with respect to the packing, thereby providing an activity-replenished desorption reactor; and
recommencing operation in the activity-replenished desorption reactor for CO₂ desorption.

47. A method for desorption of an ion-loaded solution comprising hydrogen and bicarbonate ions, the method comprising:
supplying ion-loaded solution into a desorption reactor comprising packing with immobilized enzymes provided on the packing;
operating the desorption reactor to produce a regenerated solution and a CO₂ gas by an enzymatically catalyzed dehydration reaction;
at a low enzyme activity threshold:
stopping operation in the desorption reactor; and
subjecting the desorption reactor to in situ activity replenishment, comprising:
removing the ion-loaded solution from the desorption reactor;
flowing a first solution through the desorption reactor to contact and pre-treat the packing material;
flowing a second solution comprising a functionalizing compound the desorption reactor to contact the packing material and produce a functionalized packing;
flowing a third solution comprising a crosslinker through the desorption reactor to contact the packing material and produce a crosslinker treated packing;
flowing a fourth solution comprising a linker through the desorption reactor to contact the packing material and produce a linker treated packing;
flowing a fifth solution comprising a crosslinker through the desorption reactor to contact the packing material and produce a pre-treated packing;
flowing a sixth solution comprising enzyme through the desorption reactor to contact the packing material and produce an enzyme activated packing; and
flowing a seventh solution comprising a reducing agent through the desorption reactor to contact the enzyme activated packing,
thereby providing an activity-replenished desorption reactor; and
recommencing operation in the activity-replenished desorption reactor for CO₂ desorption.

48. A method for desorption of an ion-loaded solution comprising hydrogen and bicarbonate ions, the method comprising:
supplying ion-loaded solution into a desorption reactor comprising packing with immobilized enzymes provided on the packing;
operating the desorption reactor to produce a regenerated solution and a CO₂ gas by an enzymatically catalyzed dehydration reaction;
monitoring enzyme activity of the immobilized enzymes;
at a low enzyme activity thresh old:
stopping operation in the desorption reactor; and
replenishing the enzymatic activity in situ; and
recommencing operation in the desorption reactor for CO₂ desorption using the replenished immobilized enzymes.

49. The method of claim 48, wherein the replenishing of the enzymatic activity comprises providing an enzyme replenishing solution into the packed reactor to contact the packing and provide a replenishing amount of the immobilized enzymes.

50. The method of claim 48, wherein the step of stopping operation in the desorption reactor comprises stopping flow of the ion-loaded solution into the desorption reactor.

51. The method of claim 48, wherein the enzymes are entrapped in an immobilization material.

52. The method of claim 51, wherein the immobilization material is coated onto the packing.

53. The method of claim 52, wherein the immobilization material comprises polysulfone, polysulfone grafted with polyethylene glycol, chitosan, polyacrylamide and/or alginate.

54. The method of claim 48, wherein the enzymes are bonded with an immobilization material to the surface of the packing.

55. The method of claim 48, wherein the replenishing of the enzymatic activity comprises spraying the enzyme replenishing solution comprising the enzyme and an immobilization material into the desorption reactor.

56. The method of claim 55, wherein the spraying is performed by nozzles integrated into the desorption reactor and/or by a separate spraying device.

57. The method of claim 56, wherein the nozzles are located at a top of the desorption reactor.

58. The method of claim 56, wherein the desorption reactor comprises several stacks of packing and the nozzles are at a top location of each stack.

59. The method of claim 56, wherein the nozzles are located on a side of the desorption reactor in one location or arranged along a whole length of the desorption reactor.

60. The method of claim 55, wherein the replenishing solution is provided into the desorption reactor via a liquid inlet that provided the ion-loaded solution to the desorption reactor.

61. The method of claim 48, wherein at least two of the desorption reactors are operated in parallel and only one of the desorption reactors is stopped and subjected to in situ activity replenishment at a time.

62. The method of claim 48, wherein the ion-loaded solution is a potassium carbonate solution or a sodium carbonate solution.

63. The method of claim 48, wherein the enzyme replenishing solution provides a replenished coating of immobilized enzymes onto the packing.

64. The method of claim 63, wherein the replenished coating is provided in a thickness that negligibly increases the size of the packing.

65. The method of claim 48, further comprising, before replenishing the activity, providing an immobilization material removal fluid into the desorption reactor to remove at least some deactivated material therefrom.

66. The method of claim 48, wherein replenishing the enzymatic activity in situ comprises soaking the enzyme replenishing solution for a period of time within the desorption reactor to substantially coat the packing surface.

67. The method of claim 48, further comprising, before replenishing the enzymatic activity in situ, drying the packing using heat, air circulation or circulation of CO₂ containing gas.
